(19) United States
(12) Patent Application Publication
(10) Pub. No.: US 2010/0129257 A1
(43) Pub. Date: May 27, 2010

(54) Waste treatment vessel featuring tilt mechanism and associated door arrangement

(75) Inventors: Jan K. Michalek, Pataskala, OH (US); Theodore J. Thomas, Columbus, OH (US)

Correspondence Address: ROGER A. GILCREST, 250 WEST STREET, COLUMBUS, OH, 43215-2538, US

(73) Assignee: Estech, LLC

(21) Appl. No.: 12/313,911

(22) Filed: Nov. 25, 2008

(51) Int. Cl. A61L 2/07

(52) U.S. Cl. 422/26; 422/295

(57) ABSTRACT

The present invention includes an apparatus for processing solid waste products, the apparatus comprising: (a) a rotatably mounted cylindrical vessel having a first end and a second end, at least one of the ends terminating in an opening to allow access to the interior of the vessel and to sealably close the vessel to allow pressurization thereof, the vessel adapted to move between an operating position and loading-discharge position wherein the first end is higher than the second end; (b) at least one door adapted to sealably close the vessel at at least one end, the door adapted to be completely separated from the vessel; (c) a steam inlet for injecting steam into the vessel; (d) at least one actuator for moving the vessel between an operating position and a loading-discharge position wherein the first end is higher than the second end; and each opening provided with a door that is completely detachable from the vessel; and (e) a respective attachment portion adapted to attach each door to the vessel.

### TECHNICAL FIELD

The present invention relates to the treatment of municipal solid waste (“MSW”) and the like. This invention disclosure presents a means of utilizing steam to heat and dry wet solids inside a rotating vessel with at least one entry and exit door.

### BACKGROUND OF THE INVENTION

A waste autoclave is a form of solid waste treatment that utilizes heat, steam and pressure of an industrial autoclave in the processing of waste. Saturated steam is pumped into the autoclave at elevated temperatures. The pressure in the vessel is maintained for a temperature-dependent period to allow the process to fully “cook” the waste. The autoclave process provides an effective pathogen and virus kill.

The “cooking” process causes plastics to soften and flatten, paper and other fibrous material to disintegrate into a fibrous mass, bottles and metal objects to be cleaned, and labels etc. to be removed.

Rotating waste autoclaves provide mechanical forces to further process the waste. With rotation, the cellulose fibers (in paper, cardboard, and yard wastes) are mechanically and thermally pulped, analogous to the process known as thermo-mechanical pulping in the pulp and paper industry.

After ‘cooking’, the steam flow is stopped and the pressure vented. When depressurized, the autoclave door is opened, and by rotating the drum the ‘cooked’ material can be discharged and safely and easily separated by a subsequent series of screens and recovery systems. Consequently, a waste autoclave system can serve as a functional alternative to landfills, providing benefits of recycling of clean and sterile materials recovered from municipal wastes.

A basic invention for municipal waste autoclaves is found in U.S. Pat. No. 4,540,495 (Holloway, 1985) and is now in the public domain, which is hereby incorporated herein by reference. The present invention represents an improvement over prior art apparatus and methods, such as those described in U.S. Pat. Nos. 5,540,391; 5,126,363; 5,253,764; 5,190,226; 5,361,994; 5,427,650; 5,407,809; 5,636,449; 5,655,718; 6,397,492; and 7,347,391, all of which are hereby incorporated herein by reference. PCT application PCT/US06/16773 and co-pending patent application Ser. No. 11/716,101 are also hereby incorporated herein by reference. Also incorporated herein by reference is co-pending U.S. Patent Provisional Patent Application No. 61/195,791 entitled Solid Waste Compression Loading and Waste Treatment Apparatus and Method, filed Oct. 10, 2008.

The cost performance of an autoclave in a waste treatment operation is driven predominantly by throughput, part of which is determined by the rapidity with which the vessels can be loaded and unloaded. Practitioners to date have used a variety of methods to load vessels, ranging from loose feed systems (e.g., conveyors) to light duty compression systems (compressing conveyors) to heavy-duty compression systems (compaction and load). Systems to date have not solved the core problem, which is to rapidly load an autoclave, preferably while rotating, with a designed or predetermined load of material.

In expansion of this core problem, MSW typically arrives on a “tipping” floor as a compacted mass. This compacted mass is broken apart and larger salvageable items are recovered. Waste materials expand in volume during the recovery.

In autoclave system processing, waste is moved by conveyor from the tipping floor to the processing floor. Waste may move more or less continuously after the tipping floor begins operations, while waste is charged into a vessel over a much shorter period. Thus, it is desirable to provide a “warehousing” activity and facility to store and accumulate waste over an hour or so, and then release it much more rapidly for load-out into a vessel.

Vessels are typically designed to operate within a 5-40% of full volume load range. Typically, an initial load will see significant volume reduction during vessel operations. Thus it is possible to compress the waste prior to or during the loading process, while still resulting in a partially filled vessel (on the basis of volume) after processing.

The economics of a system are driven by throughput. It is therefore desirable to operate a vessel near its upper design load, rather than near its lower design load. In order to achieve the upper design load, the load must be compressed prior to and/or during the loading process.

In addition, it is desirable to be able to conveniently, safely and quickly load a waste mass charge into a vessel, whether compressed or not, without interference from steam lines or other mechanical arrangements, such as hinges, typically associated with steam autoclave doors.

### SUMMARY OF THE INVENTION

One of the key aspects of the present invention is the use of a system that separates the doors from the vessel in such a way that the vessel is opened widely to allow the waste mass to be more readily and conveniently loaded and unloaded in the processing cycle, so as to “debottleneck” the waste compression/loading process.

This present invention provides improvements in the functionality of waste autoclave systems by increasing loading and unloading efficiency and the resultant overall throughput efficiency.

The present invention may also be used in conjunction with an environmental separation system as described in co-pending related U.S. Patent Application Ser. No. 61/123,351 (hereby incorporated herein by reference), which describes designs and practices that segregate the clean area of the exit end of the vessel from the non-clean area beginning with the tipping floor.

The use of baled wastes may also be incorporated into the loading process used with the apparatus described herein. This method, apparatus and system is described in U.S. Patent Provisional Patent Application No. 61/195,791 entitled Solid Waste Compression Loading and Waste Treatment Apparatus and Method, filed Oct. 10, 2008.

The present invention may also be used in conjunction with a waste processing apparatus and method, featuring a preferred arrangement of blades and/or acutators, as described in co-pending related U.S. Pat. No. 7,347,391.

In general terms, the invention includes an apparatus, system, waste treatment facility and method of autoclave loading and treatment.

The present invention includes several aspects as generally described below

Apparatus with Door and Tilt Mechanism

In general terms, the present invention includes an apparatus for processing solid waste products, the apparatus comprising: (a) a rotatably mounted cylindrical vessel having a first end and a second end, at least one of the ends terminating in an opening to allow access to the interior of the vessel and to sealably close the vessel to allow pressurization thereof, the vessel adapted to move between an operating position and loading-discharge position wherein the first end is higher than the second end; (b) at least one door adapted to sealably close the vessel at the at least one end, the door adapted to be completely separated from the vessel; (c) a steam inlet for injecting steam into the vessel; (d) at least one actuator for moving the vessel between an operating position and a loading-discharge position wherein the first end is higher than the second end; and each opening provided with a door that is completely detachable from the vessel, and (e) a respective attachment portion adapted to attach each door to the vessel.

It is preferred that the operating position is horizontal, or at least that the vessel be held in or returned to a substantially horizontal position immediately prior to one of the doors being opened. Once opened, it is preferred that the vessel may be tilted upward to allow for loading of the vessel if empty. Otherwise, it is preferred that, once opened, the vessel containing a charge of treated waste may be tilted downward to aid in removal of the treated waste mass, such as onto a conveyor or processing surface, or into a vehicle. Once the vessel is emptied, it is preferred that the vessel may be tilted upward to allow for re-loading of the vessel, such as from a conveyor or vehicle. Once loaded, the vessel may be returned to or held at its normal processing position(s), whether that be horizontal, a fixed angle to the horizontal, or moved through several processing positions as described in earlier patents. The vessel may be loaded and unloaded from the same end or from different ends, and the associated conveyor or vehicle lane or support, may be positioned accordingly.

In a preferred embodiment, the apparatus of the present invention additionally comprises a support mechanism separate from the vessel and adapted to support each door when completely separated from the vessel. In this regard, as used herein, reference to separation means separated without any substantial weight supportive structure attaching the door to the vessel. This support may be in any appropriate form, and typically and preferably will permit the freed vessel door to be moved completely away from the vessel opening so as to allow for any mechanism, i.e., conveyor or vehicle and the like, to have unobstructed access to the vessel opening for loading and/or unloading. Preferably, the support mechanism will comprise a clamp, tether or similar structure adapted to engage each door when separated from the vessel. This may be supported from the ground/floor or from above as the facility allows. It will be preferred that the door is held in a position that allows it to be most efficiently replaced onto the vessel opening, such as being held in position where it can be returned and aligned along the longitudinal axis of the vessel for ease of reconnection.

It is preferred that where the operating and/or loading/unloading position is horizontal, the support mechanism is adapted to move the door between a closed position engaging the vessel and an open position sufficiently away from the vessel so as to allow the first end to accept solid waste products when the vessel is in the loading-discharge position, and to allow the second end to discharge treated solid waste products when the vessel is in the loading-discharge position.

As described herein, it is preferred that the attachment means for the door be sufficient to seal the vessel against the operating temperature and pressure, while accommodating the removal of the door. For instance, in a preferred embodiment, the opening(s) and the door(s) are provided with edge flanges and wherein the respective attachment portions comprise clamps adapted to engage respective edge flanges when the doors are in the closed position. The respective attachment portions may each comprise a pair of actuators disposed aside each respective door and adapted to urge the respective clamps to engage respective edge flanges for the purpose of supporting the door. The respective attachment portions may optionally include a supporting shelf on which the door may be supported as the door is uncoupled from the vessel.

The apparatus may also include one or more loading conveyor(s) disposed so as to supply solid waste products to at least one opening end when the vessel is in the loading-discharge position. The apparatus may also include a discharge conveyor disposed so as to accept treated solid waste products from the unloading end when the vessel is in the loading-discharge position.

Method of Loading/Unloading with Door and Tilt Mechanism

The present invention also includes a method of loading an apparatus for processing solid waste products, the apparatus comprising: (a) a rotatably mounted cylindrical vessel having a first end, a second end and an interior surface, two ends each terminating in an opening to allow access to the interior of the vessel and sealably closed to allow pressurization of the vessel, the vessel adapted to move between an operating position and loading-discharge position wherein the first end is higher than the second end; (b) a steam inlet for injecting steam into at least one of the ends; (c) at least one actuator for moving the vessel between a horizontal operating position and loading-discharge position wherein the first end is higher than the second end; and the first end opening provided with a first end door and the second end opening provided with a second end door, each of which are completely detachable from the vessel, and (d) a respective attachment portion adapted to attach each door to the vessel, the method comprising: (1) placing the vessel in the loading-discharge position while the first end door has been completely detached from the vessel, and while the second end door has been closed against the second end of the vessel; (2) placing solid waste products into the first end of the vessel; (3) and moving the vessel to the horizontal operating position; and (4) closing the first end door.

A variation upon the method of the present invention is a method of unloading an apparatus for processing solid waste products, the apparatus comprising: (a) a rotatably mounted cylindrical vessel having a first end, a second end and an interior surface, two ends each terminating in an opening to allow access to the interior of the vessel and sealably closed to allow pressurization of the vessel, the vessel adapted to move between an operating position and loading-discharge position wherein the first end is higher than the second end; (b) a steam inlet for injecting steam into at least one of the ends; (c) at least one actuator for moving the vessel between a horizontal operating position and loading-discharge position wherein the first end is higher than the second end; and the first end opening provided with a first end door and the second end opening provided with a second end door, each of which are completely detachable from the vessel; and (d) a respective attachment portion adapted to attach each door to the vessel, the method comprising: (1) placing the vessel containing treated solid waste products in the horizontal operating position with the first end door and second end door in a closed position; (2) opening the second end door by completely detaching it from the vessel; (3) moving the vessel to the loading-discharge position so as to discharge the treated solid waste products from the vessel; (4) returning the vessel to the horizontal operating position; and (5) closing the second end door.

Other variations in the invention may take advantage of one- or two-opening vessels of the present invention, and the wide variety of loading and discharge methods that are possible, including (1) removing the door while the vessel is substantially horizontal followed by lowering the opened side to discharge the vessel contents, further followed by raising the opened end to above horizontal to accept a new charge of waste to be treated (this may be used in single-opening vessels), (2) having the vessel in a horizontal position following processing, opening both ends of the vessel followed by either (a) removing the treated mass by action of a ram or by action of rotation of the vessel having helical blades, or (b) raising the vessel so as to lower the discharge end, and then reattaching the discharge end door (whether in the horizontal or tilted position) followed by placing an additional charge of waste into the vessel while in, or being raised to, an angled position with the loading end raised.

It is preferred that the autoclave preferably is disposed at an angle to the horizontal for loading so as to assist in the loading of the uncompressed waste mass or bales, and it is also preferred that the autoclave is rotated during loading so as to assist in the loading of the loose waste mass or compressed bales.

The present invention may be adapted to operate with any autoclave system, such as those known and used in the art. Autoclaves that are held permanently at an angle, or those that may be tilted from the horizontal are preferred owing to the loading system featured in the present invention.

The conveyor additionally preferably comprises a conveyor belt and a mechanical forcing arrangement for rapidly and positively charging the bales into the autoclave. The autoclave most preferably is disposed at an angle to the horizontal so as to assist in the loading of the bales, and is rotated during loading so as to assist in the loading of the bales. It is preferred that the conveyor, or at least its loading terminal segment, is linear and faces directly into the autoclave in order to the speed loading.

In a preferred embodiment, the conveyor has a loading end and a discharge end, and there is a barrier disposed between the conveyor loading end and the conveyor discharge end, such that the conveyor extends through the barrier. The barrier may be part of an entire chamber holding the autoclave so as to better separate and maintain the area of the autoclave as a clean area.

In a preferred embodiment, an additional plurality of compressed bales may be prepared while the autoclave is operating. It is preferred that the plurality of compressed bales be stored at the solid waste treatment facility prior to loading, typically adjacent to or part of the building or building complex that houses the autoclave. The plurality of compressed bales next to be loaded may be stored on the conveyor mechanism prior to loading.

The binding material may be selected from the group consisting of strapping and wrapping materials, and preferably includes polyethylene and polyethylene materials.

The compressed bales are typically charged into the autoclave through application of mechanical forces, and preferably may be thrown into the autoclave or otherwise injected into the autoclave through the application of inertial forces.

The autoclave is preferably disposed at an angle to the horizontal so as to assist in the loading of the bales. Preferably, the autoclave is rotated during loading so as to assist in the loading of the compressed bales.

The invention further includes a solid waste treatment facility for storing solid waste containing a system for loading solid waste into an autoclave at a solid waste treatment facility and treating the waste, the system comprising: (a) a building containing a system for loading solid waste into an autoclave for treatment, comprising: (i) an autoclave; (ii) a baling apparatus adapted to compress the waste into a plurality of compressed bales, the bales bound by a material adapted to melt at a temperature at which the autoclave operates; and (iii) a conveyor adapted to load the bales of waste into the autoclave; and the building containing a storage area for containing a plurality of compressed bales while the autoclave is operating.

Preferably, the building comprises a tipping floor area for receipt of solid waste from trucks, as well as a storage area for the compressed bales.

The facility and process preferably allows for the waste to be compression baled on the tipping floor, thereby allowing the entire vessel area to be a clean zone, if desired. In a preferred embodiment, there may be provided a barrier between the tipping floor area and the autoclave, with the conveyor extending from the tipping floor through the barrier to the autoclave, in order to better isolate the autoclave area as a clean area.

A solid waste treatment facility for storing solid waste containing a system for loading solid waste into an autoclave at a solid waste treatment facility and treating the waste, the system comprising: (a) a building containing a system for loading solid waste into an autoclave for treatment, the system comprising: (i) an autoclave; (ii) a baling apparatus adapted to compress the waste into a plurality of compressed bales, the bales bound by a material adapted to melt at a temperature at which the autoclave operates; (iii) a conveyor adapted to load the bales of waste into the autoclave; and (iv) the building containing a storage area for containing a plurality of compressed bales while the autoclave is operating.

The building typically and preferably comprises a tipping floor area for receipt of solid waste from trucks. The solid waste treatment facility also preferably features a building comprising a storage area for the loose waste mass or compressed bales, or the waste mass may be stored elsewhere. Most preferably, the building comprises a tipping floor area for receipt of solid waste from trucks, a barrier between the tipping floor and the autoclave, the conveyor extending from the tipping floor through the barrier to the autoclave.

The present invention also includes a method of loading solid waste into an autoclave and treating solid waste therein, the method comprising: (a) providing: (i) an autoclave; and (ii) a conveyor adapted to load the bales of waste into the autoclave, the conveyor holding a plurality of compressed bales, the bales bound by a material adapted to melt at a temperature at which the autoclave operates or otherwise breaks upon the operation of the autoclave; and (b) loading the compressed bales into an autoclave; and (c) operating the autoclave to melt or break the binding material to allow the compressed bales of solid waste to be decompressed and treated in the autoclave.

The additional and preferred parameters are as otherwise described above.

### BRIEF DESCRIPTION OF THE DRAWINGS

FIG. 1 is an elevation view of a vessel and door system for processing solid waste products in accordance with one embodiment of the present invention.

FIG. 2 is a perspective view of a vessel and door system for processing solid waste products in accordance with one embodiment of the present invention.

FIG. 3 is an elevation view of a vessel and door system for processing solid waste products in accordance with one embodiment of the present invention.

FIG. 4 is a perspective view of a vessel and door system for processing solid waste products in accordance with one embodiment of the present invention.

FIG. 5 is an elevation view of a vessel and door system for processing solid waste products in accordance with one embodiment of the present invention.

FIG. 6 is a perspective view of a vessel and door system for processing solid waste products in accordance with one embodiment of the present invention.

FIG. 7 is a detailed section view of a portion of a vessel and door for processing solid waste products in accordance with one embodiment of the present invention.

FIG. 8 is a perspective view of a vessel and door system for processing solid waste products in accordance with one embodiment of the present invention.

### DETAILED DESCRIPTION OF THE PREFERRED EMBODIMENTS

In accordance with the foregoing summary, the following provides a detailed description of the preferred embodiment, which is presently considered to be the best mode thereof.

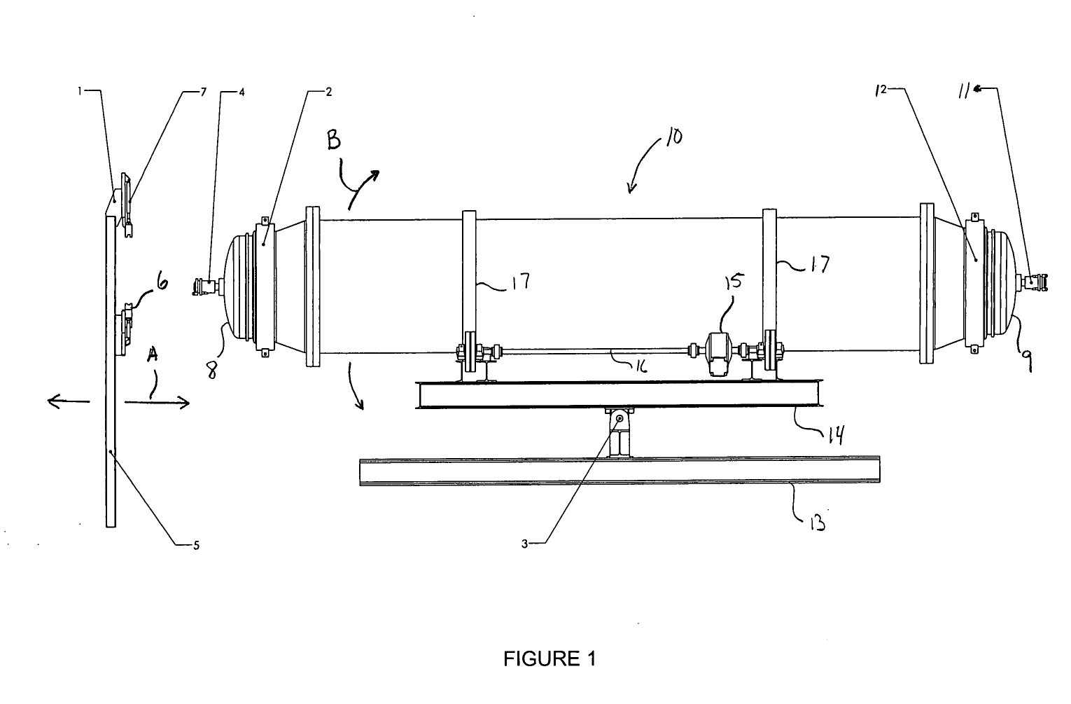

FIGS. 1-6 and 8 are views of a vessel and door system for processing solid waste products in accordance with one embodiment of the present invention. Like reference numerals are used to refer to corresponding elements and features in each of the Figures as described below.

FIG. 1 is an elevation view of a vessel and door system for processing solid waste products in accordance with one embodiment of the present invention. FIG. 1 shows a side view of the autoclave vessel 10 in normal operating position, with doors 8 and 9 attached. For simplicity, steam hoses and other details are not shown.

FIG. 1 shows door support unit 1 having door engagement clamps such as 7 that are adapted to capture and retain doors such as 8 or conversely door 9 when such an apparatus as door support unit 1 is disposed on the other end of the vessel 10 as well. FIG. 1 also shows steam inlets 4 and 11 associated respectively with doors 8 and 9. Also shown are opposed C-clamp closures 2 and 12 that are associated respectively with doors 8 and 9, and serve to attach the doors respectively to vessel 10. Vessel 10 is supported in such a way that allows it to be moved from a substantially horizontal position where it is positioned to have release and accept the separable doors 8 and 9. This may be done by any mechanical arrangement, such as for instance providing a sub-frame 14 that connects through the use of a pivot point 3 that in turn attaches to lower frame 13 that may in turn rest upon and/or be attached to a facility floor. The sub-frame 14 may support drive mechanism of any appropriate arrangement or design, such one comprising motor 15, axle 16 and gear drives 17 (that may likewise be chain or pulley drives as well).

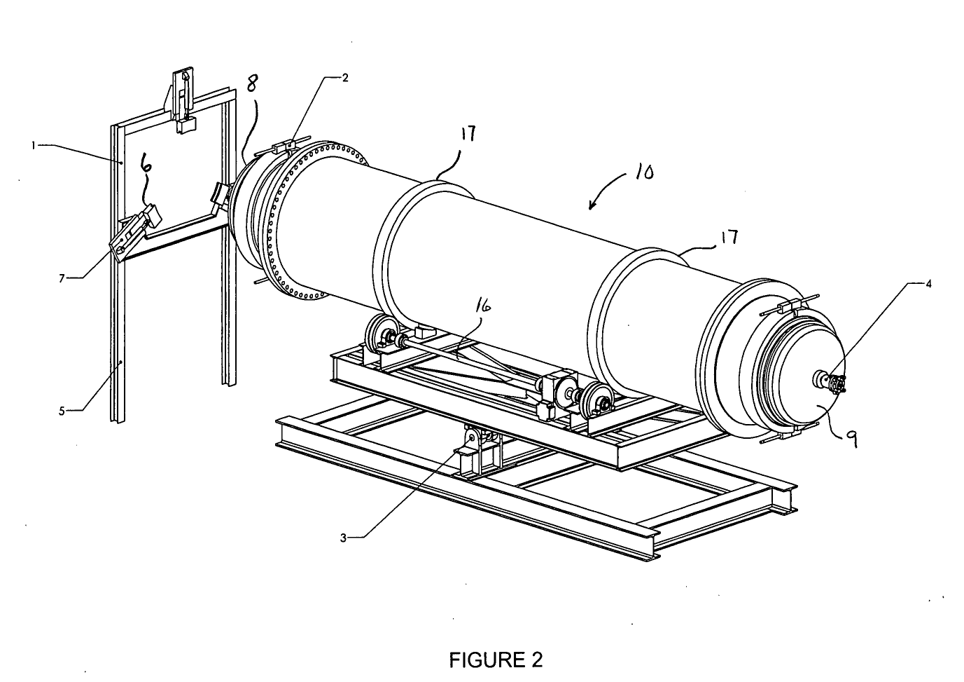

FIG. 2 is a perspective view of a vessel and door system for processing solid waste products in accordance with one embodiment of the present invention. FIG. 2 shows an isometric view of the autoclave vessel 10 in normal operating position, with doors 8 and 9 attached. For simplicity, a schematic of the door removal mechanism borne on support 5 for the left side of the vessel 10 is shown. The right side mechanism, not shown, is identical and would be in a mirror image position.

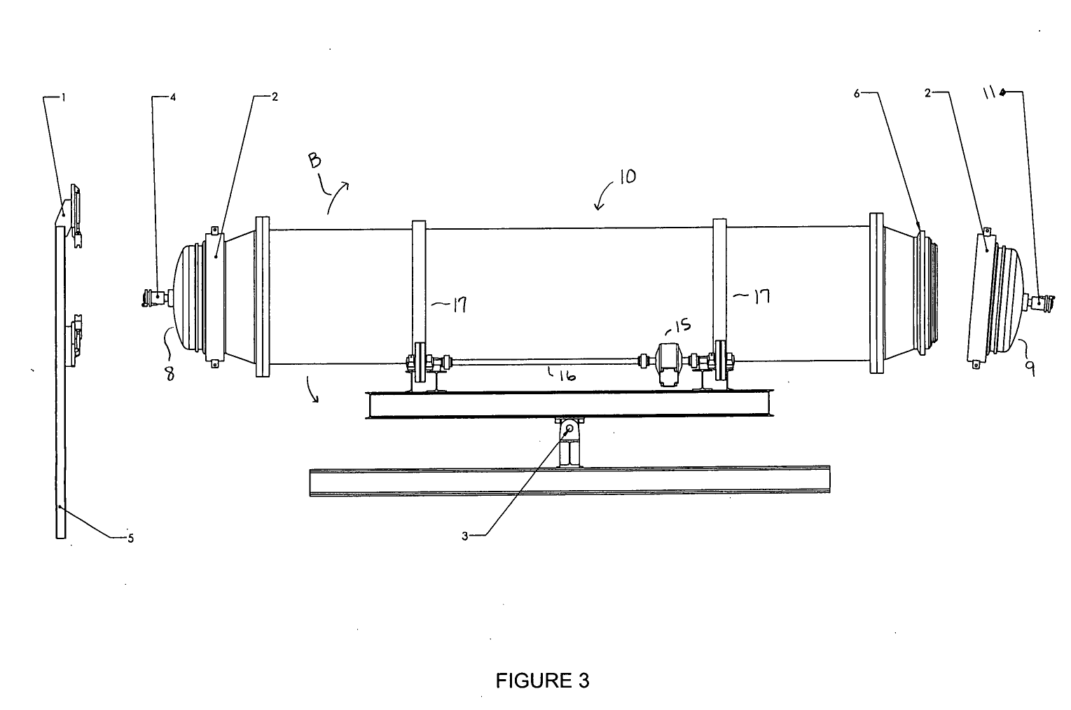

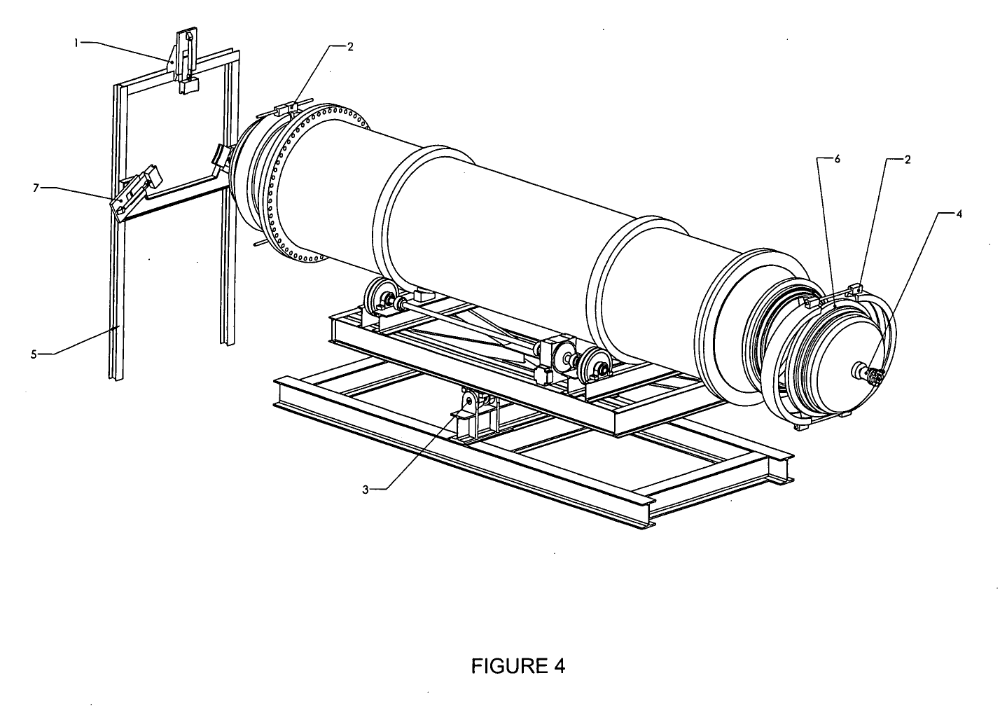

FIG. 3 is an elevation view of a vessel and door system for processing solid waste products in accordance with one embodiment of the present invention. FIG. 3 shows a side view of the autoclave vessel 10 with the door 9 removed (the door removal mechanism is not shown). This is repeated again in FIG. 4, an isometric view.

In operation, the doors, such as door 9 is adapted to be completely separated from the vessel 10 (save for the steam lines that may remain connected to the door), as show in this Figure. In the preferred embodiment, the freed door 9 may be clamped onto, supported and distanced from the vessel 10 by means of door support unit 1 as would be provided on the same side of the vessel as door 9.

FIG. 4 is a perspective view of a vessel and door system for processing solid waste products in accordance with one embodiment of the present invention. FIG. 4 shows the action of opposed C-clamps closure 2 which is but one example of the closure and seal mechanism that may be used in accordance with the present invention. One may also use screw arrangements, or any other arrangement that preferably and advantageously allows for the closure and seal, and, upon opening, the complete separation of the door(s) 8 and/or 9 from vessel 10.

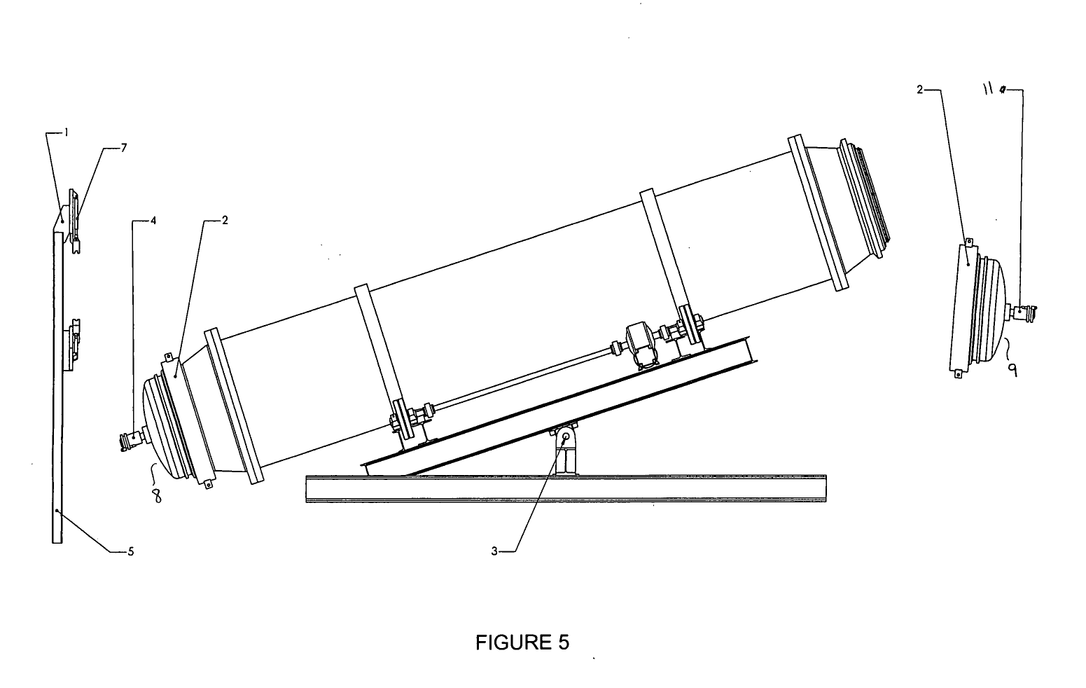

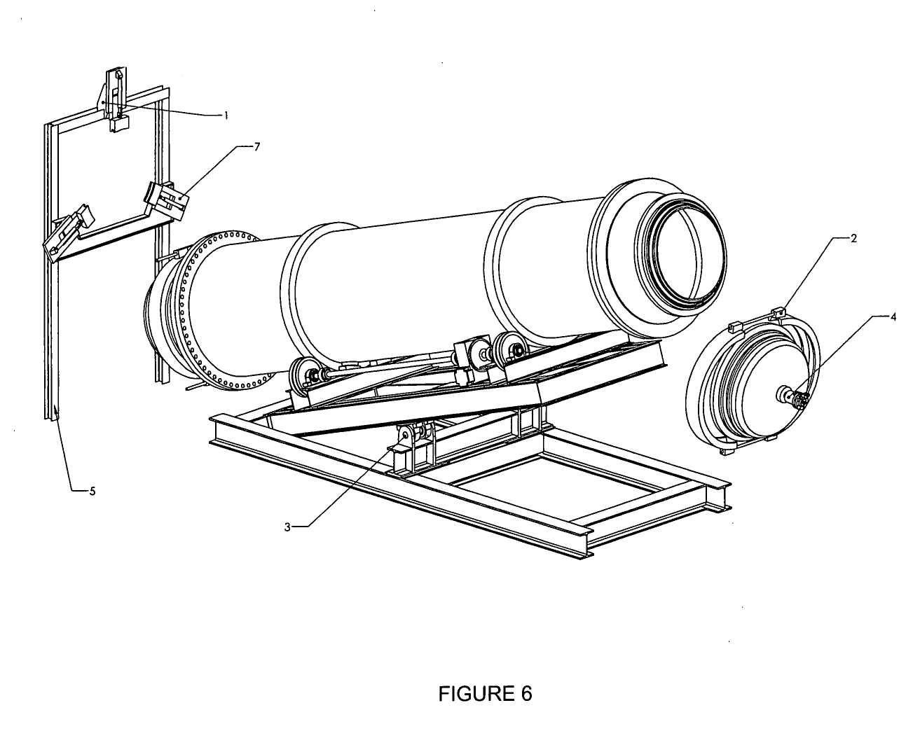

FIG. 5 is an elevation view of a vessel and door system for processing solid waste products in accordance with one embodiment of the present invention. FIG. 5 shows a side view of the autoclave vessel 10 with the door 9 removed, and the vessel 10 tilted into a loading configuration, such as may be used to load the vessel from an elevated conveyor as is known. In this instance, loading occurs over the top of the removed door and its mechanism. This is repeated again in FIG. 6, an isometric view, which is a perspective view of a vessel and door system for processing solid waste products in accordance with one embodiment of the present invention.

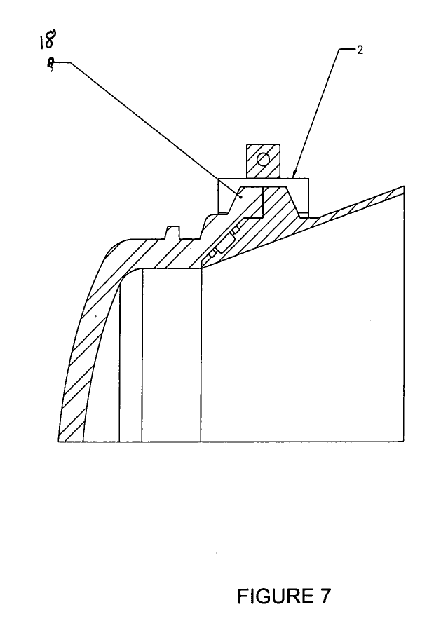

FIG. 7 is a detailed section view of a portion of a vessel and door for processing solid waste products in accordance with one embodiment of the present invention. This Figure shows a detail of the door-vessel seal using the opposed C-clamps 2 to engage door flange 18.

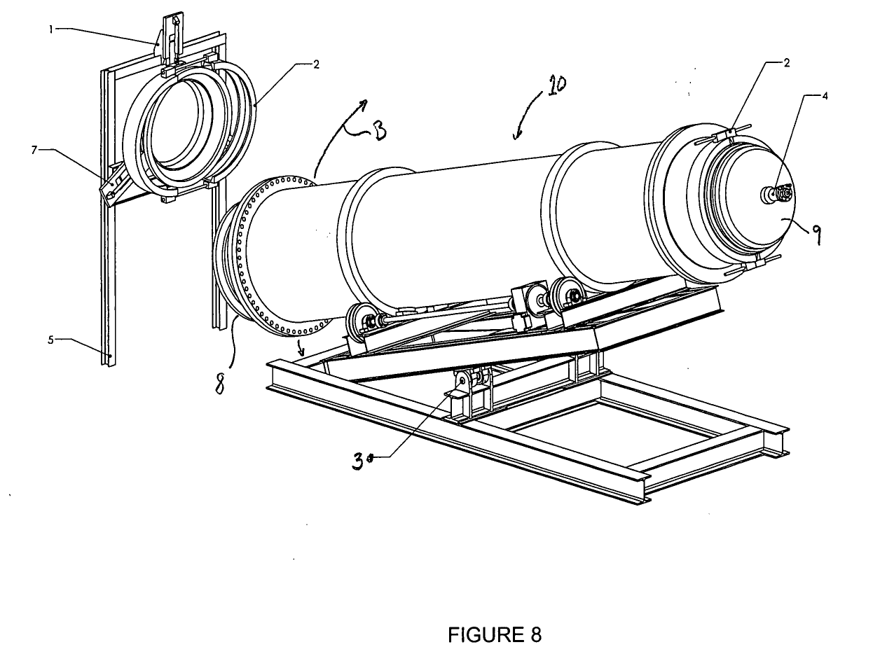

FIG. 8 is a perspective view of a vessel and door system for processing solid waste products in accordance with one embodiment of the present invention. This Figure shows a view of the autoclave vessel 10 with a door removed and stowed on the door support portion 5 with the aid of the associated engagement mechanism.

In the normal operating conditions, the door support unit 1 is separated from the vessel 10, allowing the vessel room to tilt and rotate during processing. The vessel 10 may be fixed horizontally, brought to and fixed to a fixed angle, or may be toggled back and forth such as along line B to level and process the waste load in accordance with the arrangements to do so as disclosed in the incorporated references. The door is sealed to the vessel with locks 2, such as circumferential wedge locks 2. The vessel 10 may tilt during operations around pivot 3. Steam is supplied to and removed from the vessel through rotary unions 4 and 11 that pass through the center of the frame of the door support unit 1.

To empty or fill the vessel 10, the door support unit 1 is moved into extraction position by moving it along line A which typically and preferably is parallel along the longitudinal axis of the vessel 10, wherein the weight of the door may be carried, such as on a support 5. Support member 5 may be provided with any means to move it, such as being borne on wheels or skids, or it may be fitted upon a track to allow it to efficiently be moved as desired away from vessel 10 a sufficient distance to allow for loading or unloading. Supporting clamps 7 are engaged with the door, such as door 8, and the door wedge locks 6 are opened, freeing the door(s).

The door support unit 1 then is retracted along line A, leaving the vessel 10 free to tilt to load/unload positions around pivot 3.

The contents of the vessel may be emptied once the door(s) is/are removed. This may be done either by tilting the vessel 10 to lower the discharge end with respect to the horizontal to empty the contents, such as onto a conveyor belt as shown in the incorporated references, or otherwise into vehicles, or by raising the non-discharge end with respect to the horizontal to empty the contents in similar fashion.

The loading and unloading actions discussed above may occur simultaneously at both the fill and discharge doors, or alternately the vessel may be discharged, the discharge door resealed, and then filled. If desired, other accessory apparatus, such as a ram or similar mechanism, may be used to urge the mass into and/or out of the vessel.

Door removal may occur when the vessel is horizontal. Physical dimensions of the vessel may require that the vessel door opening occurs at a level below or above horizontal to provide sufficient clearance for subsequent filling and/or discharging depending upon the ancillary loading and unloading arrangements applied.

It is apparent that while specific embodiments of the invention are disclosed, various modifications of the apparatus or parameters of the process may be made which will be within the spirit and scope of the invention. Therefore the spirit and scope of the present invention should be determined by reference to the claims below.

What is claimed is:

1. An apparatus for processing solid waste products, said apparatus comprising:
(a) a rotatably mounted cylindrical vessel having a first end and a second end, at least one of said ends terminating in an opening to allow access to the interior of said vessel and to sealably close said vessel to allow pressurization thereof, said vessel adapted to move between an operating position and loading-discharge position wherein said first end is higher than said second end;
(b) at least one door adapted to sealably close said vessel at at least one end, said door adapted to be completely separated from said vessel;
(c) a steam inlet for injecting steam into said vessel;
(d) at least one actuator for moving said vessel between an operating position and loading-discharge position wherein said first end is higher than said second end; and each said opening provided with a door that is completely detachable from said vessel; and
(e) a respective attachment portion adapted to attach each said door to said vessel.

2. An apparatus according to claim 1 wherein said operating position is horizontal.

3. An apparatus according to claim 1 additionally comprising a support mechanism separate from said vessel and adapted to support each said door when separated from said vessel.

4. An apparatus according to claim 3 wherein said support mechanism comprises a clamp adapted to engage each said door when separated from said vessel.

5. An apparatus according to claim 3 wherein said operating position is horizontal, and wherein said support mechanism is adapted to move said door between a closed position engaging said vessel and an open position sufficiently away from said vessel so as to allow said first end to accept solid waste products when said vessel is in said loading-discharge position, and to allow said second end to discharge treated solid waste products when said vessel is in said loading-discharge position.

6. An apparatus according to claim 1 wherein said openings and said doors are provided with edge flanges and wherein said respective attachment portions comprise clamps adapted to engage respective edge flanges when said doors are in the closed position.

7. An apparatus according to claim 5 wherein said respective attachment portions each comprise a pair of actuators disposed aside each respective door and adapted to urge said respective clamps to engage respective edge flanges for the purpose of supporting the door.

8. An apparatus according to claim 5 wherein said respective attachment portions include a supporting shelf on which the door may be supported as the door is uncoupled from the vessel.

9. An apparatus according to claim 1 additionally comprising a loading conveyor disposed so as to supply solid waste products to said first end when said vessel is in said loading-discharge position.

10. An apparatus according to claim 1 additionally comprising a discharge conveyor disposed so as to accept treated solid waste products from said second end when said vessel is in said loading-discharge position.

11. A method of loading an apparatus for processing solid waste products, said apparatus comprising:
(a) a rotatably mounted cylindrical vessel having a first end, a second end and an interior surface, two ends each terminating in an opening to allow access to the interior of said vessel and sealably closed to allow pressurization of said vessel, said vessel adapted to move between an operating position and loading-discharge position wherein said first end is higher than said second end;
(b) a steam inlet for injecting steam into at least one of said ends;
(c) at least one actuator for moving said vessel between a horizontal operating position and loading-discharge position wherein said first end is higher than said second end; and said first end opening provided with a first end door and said second end opening provided with a second end door, each of which are completely detachable from said vessel; and
(d) a respective attachment portion adapted to attach each said door to said vessel, said method comprising:
(1) placing said vessel in said loading-discharge position while said first end door has been completely detached from said vessel, and while said second end door has been closed against said second end of said vessel;
(2) placing solid waste products into said first end of said vessel;
(3) and moving said vessel to said horizontal operating position; and
(4) closing said first end door.

12. A method of unloading an apparatus for processing solid waste products, said apparatus comprising:
(a) a rotatably mounted cylindrical vessel having a first end, a second end and an interior surface, two ends each terminating in an opening to allow access to the interior of said vessel and sealably closed to allow pressurization of said vessel, said vessel adapted to move between an operating position and loading-discharge position wherein said first end is higher than said second end;
(b) a steam inlet for injecting steam into at least one of said ends;
(c) at least one actuator for moving said vessel between a horizontal operating position and loading-discharge position wherein said first end is higher than said second end; and said first end opening provided with a first end door and said second end opening provided with a second end door, each of which are completely detachable from said vessel; and
(d) a respective attachment portion adapted to attach each said door to said vessel, said method comprising:
(1) placing said vessel containing treated solid waste products in said horizontal operating position with said first end door and second end door in a closed position;
(2) opening said second end door by completely detaching it from said vessel;
(3) moving said vessel to said loading-discharge position so as to discharge said treated solid waste products from said vessel;
(4) returning said vessel to said horizontal operating position; and
(5) closing said second end door.
